(19) United States
(12) Patent Application Publication
(10) Pub. No.: US 2021/0020319 A1
(43) Pub. Date: Jan. 21, 2021

(54) INTEGRATED ON-LINE AND OFF-LINE INTERROGATION SYSTEM

(71) Applicant: Shanghai Rainon Pharmaceuticl Technology Co., Ltd, Shanghai (CN)

(72) Inventor: Dong LIU, Shanghai (CN)

(21) Appl. No.: 16/932,090

(22) Filed: Jul. 17, 2020

(30) Foreign Application Priority Data
Jul. 19, 2019 (CN) 201910654196.9

(51) Int. Cl. G16H 80/00; G16H 10/60

(52) U.S. Cl. CPC G16H 80/00; G16H 10/60

(57) ABSTRACT

An integrated on-line and off-line interrogation system includes a physician-seeking system, an on-line interrogation system, a banking card transaction management system, an electronic health record management system, a (on-line) physician module, and a (off-line, in-hospital) physician module. The physician-seeking system allows patients to search for physicians. Patients can select on-line real-time interrogation and appointment, or select off-line interrogation and add-on or registration, or be referred to off-line interrogation from on-line referral. Patients' image data and health records of off-line interrogation can be classified and managed using the electronic health record management system, thereby achieving convenient and paperless operation. Pairing (on-line) physicians and (off-line, in-hospital) physicians provides a basis for localization management of physicians and facilitates on-line and off-line referral. This system establishes a real-time, convenient interrogation channel between patients and physicians without spacial and temporal limitation, thereby maximizing possibility of interrogation and solving difficulty in physician seeking and queuing.

### BACKGROUND OF THE INVENTION

### 1. Technical Field

The present invention relates to on-line interrogation, and more particularly to an integrated on-line and off-line interrogation system.

### 2. Description of Related Art

In the past days when network-based communication was far from mature, people with illness had to go to hospitals personally and make registration on site before they could see physicians in face. In regions where medical resources were concentrated, patients were more concentrated, causing registration, queuing and receiving medicine to be time-consuming and troublesome. This means patients had to not only travel around for seeing a physician, but also spent considerable time for waiting. Both of these are unaffordable to aged and seriously ill patients. With maturity of network development and popularization of on-line services, it is now possible and desirable to integrate medical resources for convenient and easy use of extensive patients. Currently, practice of medical interrogation has the following problems. First, a physician can only provide his/her services during clinic hours, and has his/her idle time in the hospital wasted in vain instead of used to take care of patients not unable to visit the hospital. Second, due to imbalance of medical resources, it is usually difficult to make appointment and registration in superior hospitals. Third, physicians in different regions may have their compensations greatly different, and unsatisfying paid physicians may be less motivated. Fourth, most existing medical interrogation systems have defective connection between on-line physician services and off-line hospital services, resulting in inconsistent quality of on-line physician services. Fifth, many existing medical interrogation systems have time lag between patients' interrogation and physicians' responses, leading to poor timeliness of medical consultation. Sixth, many medical interrogation systems lack for “face-to-face” visual interrogation function, so the resulting interrogation is incomprehensive, and these systems are usually not supported by systematic electronic health record management, making on-line interrogation less integral.

### SUMMARY OF THE INVENTION

In view of the shortcoming of the prior art, the present invention discloses an integrated on-line and off-line interrogation system, which comprises a physician-seeking system, an (on-line) physician module, a (off-line, in-hospital) physician module, an on-line interrogation system, a banking card transaction management system, and an electronic health record management system. Therein, the physician-seeking system is connected with the (on-line) physician module. The (on-line) physician module and the (off-line, in-hospital) physician module are one-to-one paired. The (on-line) physician module, the banking card transaction management system and the electronic health record management system are connected with the on-line interrogation system. The (off-line, in-hospital) physician module is connected with the banking card transaction management system.

Further, the physician-seeking system comprises a physician status operation system, a physician function operation system, an on-line real-time interrogation button, and an on-line appointment interrogation opening button. Therein, physician status operation system is connected with the physician function operation system. The physician function operation system comprises an on-line real-time interrogation button and an on-line appointment interrogation opening button. The on-line real-time interrogation button and the on-line appointment interrogation opening button are connected with the on-line interrogation system.

Further, the (off-line, in-hospital) physician module comprises an on-line appointment add-on/registration system, off-line hospital outpatient check-in, off-line hospital registration, and off-line hospital interrogation. Therein, the on-line appointment add-on/registration system is connected with the off-line hospital outpatient check-in. The off-line hospital outpatient check-in is connected with the off-line hospital registration. The off-line hospital registration is connected with the off-line hospital interrogation. The off-line hospital interrogation is connected with the electronic health record management system. The off-line hospital interrogation is connected with the banking card transaction management system.

Further, the on-line interrogation system comprises an on-line real-time interrogation module, an on-line appointment interrogation module, a message module, and audio/video interrogation. Therein, the on-line real-time interrogation module and the on-line appointment interrogation module are both connected with the message module and the audio/video interrogation. The on-line real-time interrogation module and the on-line appointment interrogation module are connected with the electronic health record management system. The on-line real-time interrogation module and the on-line appointment interrogation module are connected with the banking card transaction management system.

Further, the banking card transaction management system comprises a finance management system, a transaction inquiry module, and a guarantee system.

Further, the electronic health record management system comprises an image capturing system, an image processing system, an electronic prescription module, and a physician summary module. Therein, the image capturing system is connected with the image processing system.

The physician-seeking system comprises a local three-level administrative hierarchy system, an on-line department system, a physician module, region, a hospital module, an off-line department system, and a superior department hospital star-ranking system. Therein, the local three-level administrative hierarchy system and the on-line department system are connected with the region. The hospital module is connected with the off-line department system, the superior department hospital star-ranking system, and the physician module. The off-line department system is connected with the physician module.

Further, the on-line department system comprises a two-level department hierarchy system, a hotspot department sorting system, and a department search system.

Further, the physician module comprises a name search system.

Further, the hospital module comprises GPS navigation, geographical distance sorting, and a hospital search system.

The present invention has the following benefits. First, it allows patients to use a variety of ways to search for a physician using the physician-seeking system, such as designating a physician according to indication, designating a physician according to name, or designating a physician according to a hospital. The patient can then select on-line real-time interrogation and appointment, or select off-line interrogation and add-on registration. The patient is enabled to communicate with the designated physician on-line through the on-line “face-to-face” interrogation function. This not only helps to arrest development of minor diseases in an early stage, but also allows physicians to provide on-line aid guidance for emergency. The system can further used for appointment of off-line hospital interrogation, so as to free patients from harassment of difficult registration. The image data uploaded by patients and health records of off-line interrogation can be classified and managed using the electronic health record management system, and thus become convenient to review, thereby achieving paperless operation and improving integrity of on-line interrogation. Moreover, pairing (on-line) physicians and (off-line, in-hospital) physicians in a one-to-one manner provides a basis for localization management of physicians and facilitates on-line and off-line referral, thereby preventing inconsistent quality of on-line physician services that decreases patients' confidence. This system establishes a real-time, convenient interrogation channel between patients and physicians that is not subject to spacial and temporal limitation, thereby maximizing possibility of interrogation and solving patients' problems about difficulty in physician seeking and queuing. In addition, physicians may get additional income through on-line interrogation, increasing physician's working motivation through market mechanism.

### BRIEF DESCRIPTION OF THE DRAWINGS

FIG. 1 is a structural diagram of an integrated on-line and off-line interrogation system according to the present invention;

FIG. 2 is a structural diagram of a physician operating system according to the present invention;

FIG. 3 is a structural diagram of a physician-seeking system according to the present invention;

FIG. 4 is a structural diagram of a hospital module according to the present invention; and

FIG. 5 is a structural diagram of an on-line department system according to the present invention.

### DETAILED DESCRIPTION OF THE INVENTION

### Embodiment 1

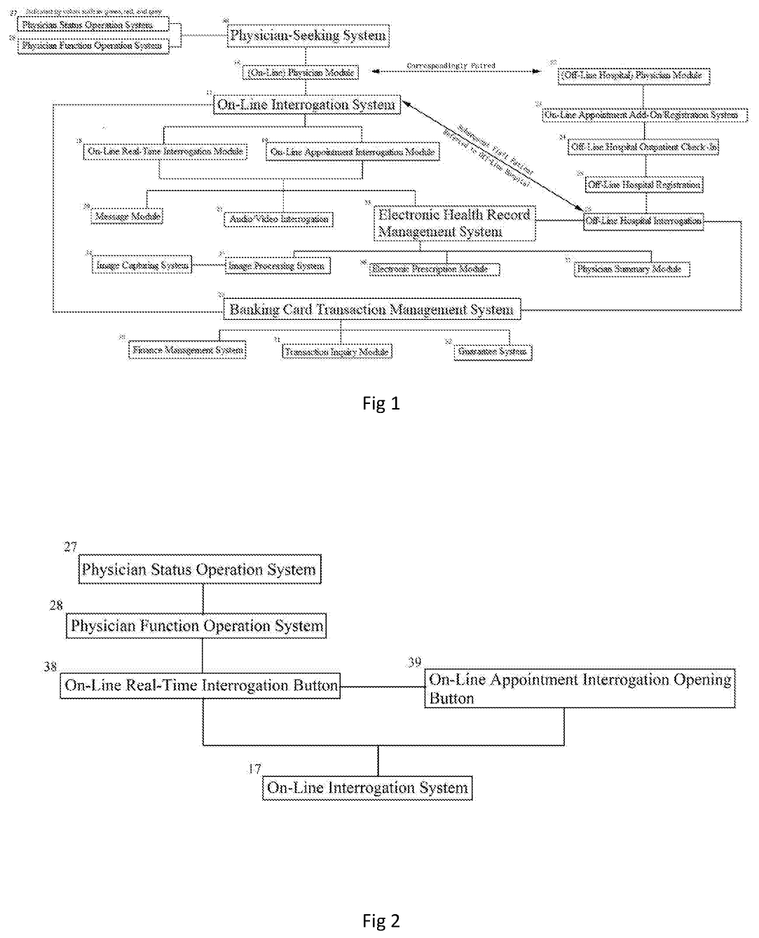

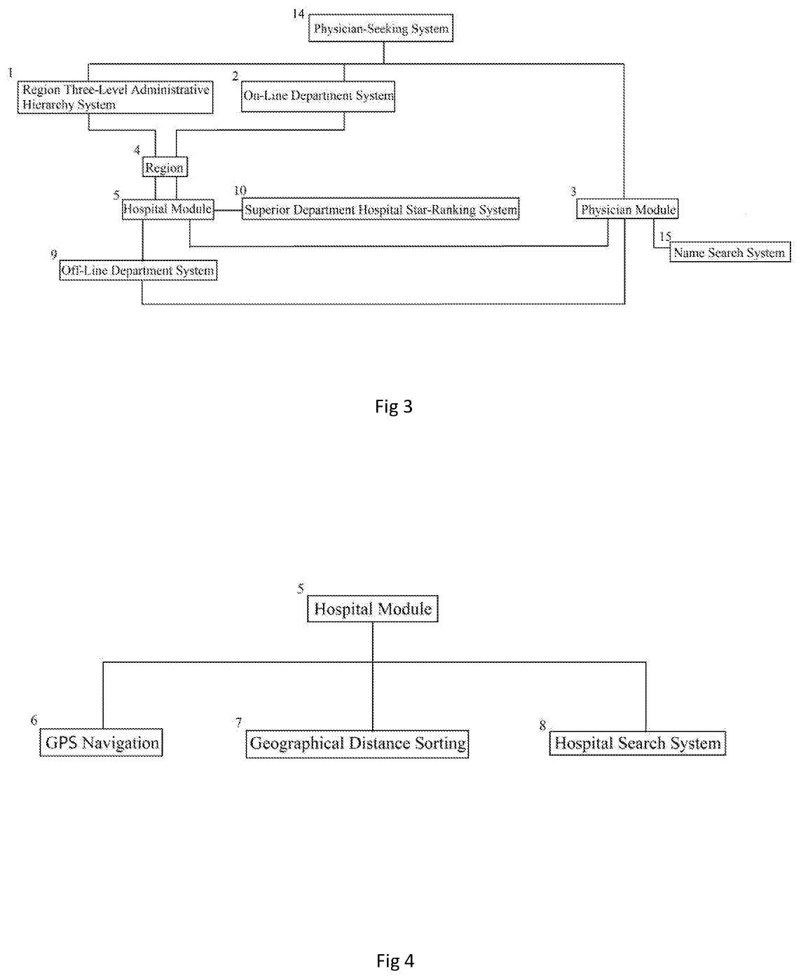

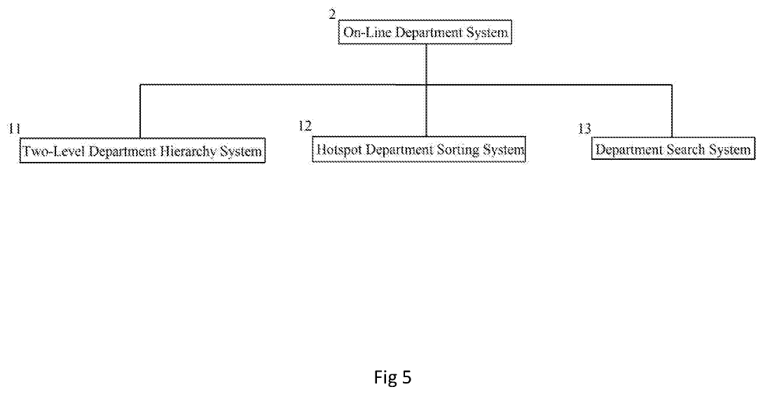

As shown in FIG. 1, FIG. 2 and FIG. 3, the present invention discloses an integrated on-line and off-line interrogation system, which comprises a physician-seeking system 40, a (on-line) physician module 16, a (off-line, in-hospital) physician module 22, an on-line interrogation system 17, a banking card transaction management system 29, and an electronic health record management system 33. Therein, the physician-seeking system 40 is connected with the (on-line) physician module 16. The (on-line) physician module 16 and the (off-line, in-hospital) physician module 22 are one-to-one paired. The (on-line) physician module 16, the banking card transaction management system 29 and the electronic health record management system 33 are connected with the on-line interrogation system 17. The (off-line, in-hospital) physician module 22 is connected with the banking card transaction management system 29.

Further, the physician-seeking system 40 comprises a physician status operation system 27, a physician function operation system 28, an on-line real-time interrogation button 38, and an on-line appointment interrogation opening button 39. Therein, the physician status operation system 27 is connected with the physician function operation system 28. The physician function operation system 28 comprises an on-line real-time interrogation button 38 and the on-line appointment interrogation opening button 39. The on-line real-time interrogation button 38 and the on-line appointment interrogation opening button 39 are connected with the on-line interrogation system 17.

Further, the (off-line, in-hospital) physician module 22 comprises an on-line appointment add-on/registration system 23, off-line hospital outpatient check-in 24, off-line hospital registration 25, and off-line hospital interrogation 26. Therein, the on-line appointment add-on/registration system 23 is connected with the off-line hospital outpatient check-in 24. The off-line hospital outpatient check-in 24 is connected with the off-line hospital registration 25. The off-line hospital registration 25 is connected with the off-line hospital interrogation 26. The off-line hospital interrogation 26 is connected with the electronic health record management system 33. The off-line hospital interrogation 26 is connected with the banking card transaction management system 29.

Further, the on-line interrogation system 17 comprises an on-line real-time interrogation module 18, an on-line appointment interrogation module 19, a message module 20, and audio/video interrogation 21. Therein, the on-line real-time interrogation module 18 and the on-line appointment interrogation module 19 are both connected with the message module 20 and the audio/video interrogation 21. The on-line real-time interrogation module 18 and the on-line appointment interrogation module 19 are connected with the electronic health record management system. The on-line real-time interrogation module 18, the on-line appointment interrogation module 19 are connected with the banking card transaction management system 29.

Further, the banking card transaction management system 29 comprises a finance management system 30, a transaction inquiry module 31, and a guarantee system 32.

Further, the electronic health record management system 33 comprises an image capturing system 34, an image processing system 35, an electronic prescription module 36, and a physician summary module 37. Therein, the image capturing system 34 is connected with the image processing system 35.

The physician-seeking system 14 comprises a local three-level administrative hierarchy system 1, an on-line department system 2, a physician module 3, region 4, a hospital module 5, an off-line department system 9, and a superior department hospital star-ranking system 10. Therein, the local three-level administrative hierarchy system 1, and the on-line department system 2 are connected with the region 4. The hospital module 5 is connected with the off-line department system 9, the superior department hospital star-ranking system 10, and the physician module 3. The off-line department system 9 is connected with the physician module 3.

Further, the on-line department system 2 comprises a two-level department hierarchy system 11, a hotspot department sorting system 12, and a department search system 13.

Further, the physician module 3 comprises a name search system 15.

Further, the hospital module 5 comprises GPS navigation 6, geographical distance sorting 7, and a hospital search system 8.

Operation of the present invention is described below. After logging in the system, a patient may search for a hospital of a designated level, such as a province level, a city level, or a county level, in a designated region using the three-level administrative hierarchy system 1. After navigating into the hospital module 5, the user may search for a route to a target hospital using the GPS navigation 6, or select a hospital nearby using the geographical distance sorting 7, or search a hospital by name directly, or navigate to the superior department hospital star-ranking system 10 to check the hospital ranking with respect to a particular department so as to make selection from available high-end medical resources. After focusing on a certain hospital in the search step, the off-line department system of that hospital is accessible, where department physicians corresponding to the physician module can be reviewed. Alternatively, a patient may enter the on-line department system directly, and search for the corresponding hospital using the two-level department hierarchy system 11 or the hotspot department sorting system 12. It is also possible for a patient to search the department in need using the department search system 13 directly. In the physician module 3, a patient may use the name search system 15 to conduct search to check the employer and department of the searched physician.

The physician may log in the physician-seeking system 40 to set his/her status using one of different colors, such as green, red, and grey, to show he/she is now available, busy or offline. The physician may in the physician function operation system 28 activate the on-line real-time interrogation button 38 or the on-line appointment interrogation opening button 39 and thereby enter the (on-line) physician module 16 to start on-line interrogation. He/she may also perform operation of the on-line appointment add-on/registration 23 in the (off-line, in-hospital) physician module 22. In the on-line interrogation system 17, he/she may select the on-line real-time interrogation module 18 or the on-line appointment interrogation module 19. Interrogation may be realized using the audio/video interrogation 21, or by receiving a message from a patient through the message module 20 and then replying to the message. The physician may make appointment add-on/registration for a patient in the (off-line, in-hospital) physician module 22, so that when arriving at the hospital, the patient may do on-site registration immediately for face-to-face off-line hospital interrogation 26. The patient may go to the hospital to perform the off-line hospital interrogation 26 directly after on-line consultation.

The data and information generated by either the on-line interrogation system 17 or the off-line hospital interrogation 26 are handled by the electronic health record management system 33. The images and video messages collected by the image capturing system 34 are processed by the image processing system 35 and then stored. The prescriptions written by the physician may be stored in the electronic prescription module 36, and the post-therapy conclusion may be entered into the physician summary module 37 for preservation.

The banking card transaction management system 29 may be used by both physicians and patients. The patient may deposit money for on-line interrogation, and check his/her transactions in the transaction inquiry module 31. The physician may use the transaction inquiry module 31 to check his/her income. For preventing irresponsible interrogation from patients and ensuring proper performance of interrogation, patients will be required to submit guarantee into the guarantee system 32.

Parts in the system that are not described herein are known in the art.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. An integrated on-line and off-line interrogation system, being characterized in: comprising a physician-seeking system (40), a (on-line) physician module (16), a (off-line, in-hospital) physician module (22), an on-line interrogation system (17), a banking card transaction management system (29), and an electronic health record management system (33), wherein the physician-seeking system (40) is connected with the (on-line) physician module (16), and the (on-line) physician module (16) and the (off-line, in-hospital) physician module (22) are one-to-one paired, in which the (on-line) physician module (16), the banking card transaction management system (29) and the electronic health record management system (33) are connected with the on-line interrogation system (17), while the (off-line, in-hospital) physician module (22) is connected with the banking card transaction management system (29).

2. The integrated on-line and off-line interrogation system of claim 1, wherein the physician-seeking system (40) comprises a physician status operation system (27), a physician function operation system (28), an on-line real-time interrogation button (38), and an on-line appointment interrogation opening button (39), in which the physician status operation system (27) is connected with the physician function operation system (28), and the physician function operation system (28) comprises a on-line real-time interrogation button (38) and an on-line appointment interrogation opening button (39), in which the on-line real-time interrogation button (38) and the on-line appointment interrogation opening button (39) are connected with the on-line interrogation system (17).

3. The integrated on-line and off-line interrogation system of claim 1, wherein the (off-line, in-hospital) physician module (22) comprises an on-line appointment add-on/registration system (23), off-line hospital outpatient check-in (24), off-line hospital registration (25), and off-line hospital interrogation (26), the on-line appointment add-on/registration system (23) being connected with the off-line hospital outpatient check-in (24), and the off-line hospital outpatient check-in (24) being connected with the off-line hospital registration (25), the off-line hospital registration (25) being connected with the off-line hospital interrogation (26), the off-line hospital interrogation (26) being connected with the electronic health record management system (33), and the off-line hospital interrogation (26) being connected with the banking card transaction management system (29).

4. The integrated on-line and off-line interrogation system of claim 1, wherein the on-line interrogation system (17) comprises an on-line real-time interrogation module (18), an on-line appointment interrogation module (19), a message module (20), and audio/video interrogation (21), in which the on-line real-time interrogation module (18) and the on-line appointment interrogation module (19) are both connected with the message module (20) and the audio/video interrogation (21), and the on-line real-time interrogation module (18) and the on-line appointment interrogation module (19) are connected with the electronic health record management system, while the on-line real-time interrogation module (18) and the on-line appointment interrogation module (19) are connected with the banking card transaction management system (29).

5. The integrated on-line and off-line interrogation system of claim 1, wherein the banking card transaction management system (29) comprises a finance management system (30), a transaction inquiry module (31), and a guarantee system (32).

6. The integrated on-line and off-line interrogation system of claim 1, wherein the electronic health record management system (33) comprises an image capturing system (34), an image processing system (35), an electronic prescription module (36), and a physician summary module (37), in which the image capturing system (34) is connected with the image processing system (35).

7. A physician-seeking system (14), being characterized in comprising a local three-level administrative hierarchy system (1), an on-line department system (2), a physician module (3), region (4), a hospital module (5), an off-line department system (9), and a superior department hospital star-ranking system (10), wherein the local three-level administrative hierarchy system (1) and the on-line department system (2) are connected with the region (4), and the hospital module (5) is connected with the off-line department system (9), the superior department hospital star-ranking system (10), and the physician module (3), while the off-line department system (9) is connected with the physician module (3).

8. The physician-seeking system of claim 7, wherein the on-line department system (2) comprises a two-level department hierarchy system (11), a hotspot department sorting system (12), and a department search system (13).

9. The physician-seeking system of claim 7, wherein the physician module (3) comprises a name search system (15).

10. The physician-seeking system of claim 7, wherein the hospital module (5) comprises GPS navigation (6), geographical distance sorting (7), and a hospital search system (8).
